(19) United States
(12) Patent Application Publication
(10) Pub. No.: US 2024/0384360 A1
(43) Pub. Date: Nov. 21, 2024

(54) Compositions and Methods of Detecting Analytes

(71) Applicant: 3M INNOVATIVE PROPERTIES COMPANY, Saint Paul, MN (US)

(72) Inventors: Gregory W. Sitton, Lansing, MI (US); Neil Percy, Lansing, MI (US)

(21) Appl. No.: 18/688,702

(22) PCT Filed: Jul. 1, 2022
(86) PCT No.: PCT/IB22/56142

§ 371 Date: Mar. 1, 2024

Related U.S. Application Data
(60) Provisional application No. 63/239,436, filed on Sep. 1, 2021.

(51) Int. Cl. C12Q 1/70; C12Q 1/6806; C12Q 1/6848; C12Q 1/6851

(52) U.S. Cl. CPC C12Q 1/701; C12Q 1/6848; C12Q 1/6851; C12Q 1/6806

(57) ABSTRACT

An aqueous composition, for example a composition for use as a lysis buffer, comprising zirconium oxide particles, a surfactant at a concentration greater than or equal to 0.005% (mass/volume), an organic, iron-chelating reagent having a first affinity constant greater than or equal to 10^{4.2} with respect to ferric iron and a second affinity constant less than 10^{3.8} with respect to magnesium (as determined in 20° C. deionized water at pH 8.45), and a buffer. The aqueous composition has a pH no less than 7.7 and less than 8.45, more particularly 7.8-8.3, in all cases when measured at 20° C. Methods of using the composition and kits comprising components of the aqueous compositions.

### BACKGROUND

U.S. Pat. No. 10,604,787 describes an aqueous lysis buffer composition (i.e., a composition that is contacted with cells to lyse the cells and release their nucleic acid) for eliminating sample inhibition in an isothermal nucleic acid amplification reaction. The composition comprises an organic iron-chelating reagent, ferric iron, a non-ionic surfactant at a concentration greater than or equal to 0.005% (mass volume), and 2-hydroxypropane-1,2,3-tricarboxylate, the 2-hydroxypropane-1,2,3-tricarboxylate and the organic iron-chelating reagent being distinct molecules. The aqueous composition has a pH of about 8.45-8.85.

US2019/0112637 discloses a nucleic acid amplification method that involves the use of a lysis buffer. The lysis buffer can include ferric ion and can further comprise a reagent selected from a group consisting of a nanoparticle dispersion stabilizer, a non-ionic surfactant having a hydrophilic-lipophilic balance of about 11 to about 16, polyvinylpyrrolidone, magnesium sulfate heptahydrate, a fluorosurfactant, an indicator dye, and a combination of two or more of said reagents. The lysis buffer is reported to have a pH of about 9.8 to 10.5 at 25° C.

U.S. Pat. No. 10,619,189 discloses an aqueous composition for eliminating sample inhibition in a nucleic acid amplification reaction that comprises a plurality of zirconium oxide particles, a non-ionic surfactant at a concentration greater than or equal to 0.005% (mass/volume) an organic iron-chelating reagent, and a nanoparticle dispersion stabilizer, polyvinylpyrrolidone, or both. The composition has a pH of about 8.45-8.85.

U.S. Pat. No. 5,693,517 discloses reagents and methods sterilizing a reverse transcription reaction contaminated with nucleic acids generated from a previous reverse transcription/amplification reaction that resulted from mixing conventional and unconventional nucleoside triphosphates. After sterilization, the nucleic acids that are to be amplified can be incubated in a liquid that includes Tris-HCl (pH 8.3), KCl, and EDTA, Tris-HCl, (pH 8.3), KCl, DTT, and MnCl₂, and bicine KOAc, and Mn(OAc)₂ (pH 7.97). This liquid, however, is not a lysis buffer and the disclosure does not relate to reducing sample inhibition by matrix compounds.

### SUMMARY OF THE DISCLOSURE

The above summary of the present disclosure is not intended to describe each disclosed embodiment or every implementation of the present disclosure. The description that follows more particularly exemplifies illustrative embodiments. In several places throughout the application, guidance is provided through lists of examples, which examples may be used in various combinations. In each instance, the recited list serves only as a representative group and should not be interpreted as an exclusive list.

### DETAILED DESCRIPTION OF ILLUSTRATIVE EMBODIMENTS

In this application, terms such as “a,” “an,” and “the” are not intended to refer to only a singular entity but include the general class of which a specific example may be used for illustration. The terms “a.” “an,” and “the” are used interchangeably with the phrases “at least one” and “one or more.” The phrases “at least one of” and “comprises at least one of” followed by a list refers to any one of the items in the list and any combination of two or more items in the list.

Terms such as “common,” “commonly,” “often,” “frequent,” and “frequently” are used to refer to features that are typically employed in the invention, but unless otherwise indicated are not meant to imply that the features so described were known or common before this disclosure.

Unless otherwise noted, all pH values in this disclosure and the appended claims refer to a pH value that is measured at 20° C.

Microorganisms or viruses, particularly those found in food samples, can be detected by way of molecular methods. Such methods, for example those that are used with the 3M Molecular Detection System (3M Company, St. Paul, MN, USA), involve contacting the sample, optionally after incubation, with an aqueous buffer liquid. The buffer liquid is a lysis buffer that lysis the cells to release nucleic acids from the cells. After contact with the lysis buffer, amplification of the nucleic acid can be carried out.

U.S. Pat. No. 10,604,787, U.S. Pat. No. 10,619,189, and US2019/0112637 disclose that zirconium oxide particles may be added to lysis buffers to reduce or eliminate the effects of the food matrix, that is, the chemical compounds from the food sample that are present in the sample that may interfere with the amplification or detection of the nucleic acid. Tris, a cationic buffer having an amine, is the preferred buffering agent in those disclosures, which indicate that the buffers must have a pH of 8.45-8.85, which is the buffering region of the tris buffer.

This disclosure recognizes several problems with the prior art lysis buffers. First, some samples may not have adequate stability at the 8.45-8.85 pH range that is disclosed in the aforementioned references but may be more stable at a lower pH range, i.e., one that is that is closer to neutral. Second, carbon dioxide in the air can act as an acid (by becoming carbonic acid when it contacts or is dissolved in an aqueous liquid), thus neutralizing the relatively high pH of the prior art buffer. This effect may be somewhat less at lower pH. Third, the amount of anionic or cationic buffer, such as tris buffer, that can be used in the aqueous liquids is somewhat limited because it can interfere with the nucleic acid amplification or detection processes. Thus, the buffering capacity of the prior-art buffers is low. The low buffering capacity limits the types of food matrices that can be tested, because some food matrices may be very acidic or basic such that the prior art buffers cannot convert them to an acceptable pH for amplification. Fourth, it would be advantageous to have a faster time to result even without regard to pH.

In brief, a solution to these and other problems lies in an aqueous composition comprising: zirconium oxide particles, a surfactant at a concentration greater than or equal to 0.005% (mass/volume), an organic, iron-chelating reagent having a first affinity constant greater than or equal to 10^{4.2} with respect to ferric iron and a second affinity constant less than 10^{3.8} with respect to magnesium, wherein the first affinity constant and the second affinity constant are determined in 20° C. deionoized water at pH 8.45, and a buffer, the buffer optionally having a concentration of 40 mM or greater, further optionally having a concentration of 40 mM to about 200 mM, and still further optionally having a concentration of 40 mM to 150 mM. The aqueous composition has a pH no less than 7.7 and less than 8.45, more particularly 7.8-8.3, in all cases when measured at 20° C. The aqueous composition is typically a lysis buffer, e.g. a lysis buffer composition.

A solution also lies in a method of amplifying nucleic acids comprising a) contacting an aqueous composition as described herein with a composition comprising a microorganism or a virus to form a mixture; b) lysing the microorganism or the virus in the mixture to form a lysed mixture; and c) subjecting at least a portion of the lysed mixture to a nucleic acid amplification process.

A solution also lies in a kit, comprising a plurality of zirconium oxide particles; a non-ionic surfactant; an organic, iron-chelating reagent having a first affinity constant greater than or equal to 10^{4.2} with respect to ferric iron and a second affinity constant less than 10^{3.8} with respect to magnesium, wherein the first affinity constant and the second affinity constant are determined in 20° C. deionoized water at pH 8.45; a buffer, the buffer having a buffering region at 20° C. that extends from a pH of 7.8 or lower to a pH of 8.2 or higher. The components in the kit can be provided as dry components to be dissolved in water by the user, for example to form an aqueous composition as described herein. Alternatively, one or more of the components of the kit can be dissolved in water in the kit, and the dry components, if any, added later.

### Aqueous compositions

The aqueous composition may be a solution or a dispersion. When a dispersion, the zirconium oxide particles are typically dispersed in the composition. The zirconium oxide particles are nanoparticles in some embodiments. In particular embodiments, the zirconium oxide particles have a mean particle size of no more than 500 nm, more particularly no more than 250 nm, and even more particularly no more than 100 nm, in each case as measured by photon correlation spectroscopy as further described herein. The zirconium oxide particles optionally have a surface area (in units of m²/L) of at least 10, such as 10-600, particularly 25-600, more particularly 50-600, even more particularly 100-600, still more particularly 200-600, even still more particularly 300-600, and most particularly 400-600.

In each case, particle size can be measured by photon correlation spectroscopy (PCS) according to the method described under the “Test Methods” section of U.S. Pat. No. 864,710. Specifically, a PCS instrument, such as a Zeta Sizer-Nano Series, Model ZEN 3600 can be equipped with a red laser (633 nm wavelength) can be used. Samples are placed into a 1 cm square cuvette to an appropriate liquid depth, such as 10-14 mm. The liquid depth will depend on the dimensions of the instrument being used. Cuvettes are then placed into the instrument and equilibrated at 25° C. Instrument parameters can be set as follows: dispersant refractive index: 1.3330, dispersant viscosity: 0.8872 mPa-sec, material refractive index: 2.10, and material absorption value 0.10 units. The instrument size-measurement procedure can then be run according to the instrument's instruction manual. Most PCS instruments will automatically adjust their laser-beam position and attenuator setting to obtain the best measurement of particle size, but if those are not automatically adjusted by the instrument being used then they can be optimized according to the instrument's instruction manual or according to standard instrument optimization techniques to obtain the best measurement (e.g., most repeatable measurement, best signal to noise ratio, etc.) of the particle size. In many cases, measuring the particle size is not required because commercially available zirconium oxide particles having a labeled particle size (e.g., that was measured by the manufacturer) are available and in general the manufacturer's representation of the particle size (e.g., on a product label) can be relied upon.

A stabilizer can optionally be added to stabilize any of the aforementioned zirconium oxide particles. Most commonly, the stabilizer is citric acid or a salt thereof, such as potassium citrate, ferric ammonium citrate, or the like. Other stabilizers may be used so long as they do not interfere with the amplification or detection of nucleic acid. In some cases, no stabilizer is required because some zirconium oxide particles can form a stable dispersion at the requisite pH values even without a stabilizer.

In any of the aforementioned cases, the pH (when measured at 20° C.) can be greater than 7.7 and less than 8.45. In particular, the pH (when measured at 20° C.) can be greater than 7.7, greater than 7.8, greater than 7.9, or greater than 8.0. In particular, the pH (when measured at 20° C.) can be less than 8.45, less than 8.4, less than 8.3, or less than 8.2. Most commonly, and most particularly, the pH is 7.8-8.3.

The buffer particularly comprises at least one zwitterionic compound, meaning that the compound is present in a zwitterionic form at the pH of the composition. Without being bound by theory, the inventors hypothesize that cationic, anionic, or nonionic buffers can coordinate with nucleic acid strands, thus interfering with the amplification or detection of the microorganisms in the sample. A particularly useful zwitterionic compound is bicine. The buffer is therefore most particularly bicine.

The buffer, particularly the zwitterionic buffer, and most particularly bicine, can have any suitable concentration but usually has a concentration of 40 mM or greater, more particularly 40 mM to 200 mM, still more particularly 40 mM to 150 mM and even more particularly 50 mM to 150 mM. These concentrations of buffer offer a higher buffering capacity than what can be obtained with cationic or anionic buffers such as tris, which will interfere with the nucleic acid amplification or detection processes at high concentrations.

The iron-chelating reagent has a first affinity constant greater than or equal to 10^{4.2} with respect to ferric iron and a second affinity constant less than 10^{3.8} with respect to magnesium. The first affinity constant and the second affinity constant are determined in 20° C. deionized water at pH 8.45. Thus, the iron-chelating reagent has a greater affinity to ferric iron than to magnesium. The iron-chelating reagent is typically an organic iron-chelating reagent, which means that the iron-chelating agent is an organic compound; this is not intended to mean that an organic iron-chelating compound only chelates organic iron compounds.

Any suitable organic, iron-chelating reagent can be used. Most typically, the organic, iron-chelating reagent comprises ethylene glycol-bis(2-aminoethylether)-N,N,N′,N′-tetracetic acid (EGTA), N,N′,N′,N′-tetrakis(2-pyridinylmethyl)ethan-1,2-diamine, 1,2-bis(O-aminophenoxy)ethane-N,N′,N′-tetracetic acid, N-(2-hydroetoxyethyl) ethylenediamine-N,N′,N′-triacetic acid, a salt of any of the foregoing, or a hydrate of any of the foregoing. Most commonly, a salt, such as a sodium or potassium salt, or a mixed sodium/potassium salt, and particularly a potassium salt, of the organic, iron-chelating reagent is used. EGTA is most commonly employed, and most particularly a potassium salt of EGTA.

Optionally, in any herein mentioned embodiment, the composition can require ferric iron. When included, the ferric iron typically has a concentration from 50-385 micromolar, such as at least 110 micromolar, at least 165 micromolar, at least 220 micromolar, at least 275 micromolar, or at least 330 micromolar; in each case the maximum concentration can be 385 micromolar. When ferric iron is included, the molar ratio of the ferric ion in the ferric iron to organic iron-chelating reagent is typically 0.04 to 0.28, more particularly 0.14 to 0.18.

The at least one non-ionic surfactant can be any suitable non-ionic surfactant that provides a stable formulation that, for example, does not precipitate components that are intended to be in solution, suspends components that are intended to be suspended, etc., for a commercially acceptable amount of time after the composition is made. Particularly useful non-ionic surfactants include those that have a hydrophilic-lipophilic balance (HLB) of 11 to 16. This HLB range facilitates the activity of DNA polymerases that are used in nucleic acid amplification, such as PCR and LAMP. Non-limiting examples of particular non-ionic surfactants that can be used include those available under the TRITON trade designation, such as TRITON X-100, TRITON X-114, TRITON X-405, TRITON X-101, and the like, sorbitan fatty acid ester, polyoxyethylene sorbitan fatty acid ester (such as those available under the TWEEN trade designation), polyoxyethylene alkyl esters (such as those available under the BRIJ trade designation), nonylphenol, lauryl alcohol, polyethylene glycol, polyoxyethylene-polyoxypropylene block copolymers, polyoxyethylene alkyl amine, polyoxyethylene fatty acid bispheyl ether, and flourosurfactants (such as those available under the NOVEC trade designation from 3M Company St. Paul MN USA).

The non-ionic surfactant can be present in any suitable concentration, for example, concentrations that meet one or more of the abovementioned criteria or other criteria as required by the particular end use. Typically, concentrations of 0.005% (w/v) to 0.3% (w/v), such as 0.01%-0.3% (w/v), are employed.

Magnesium ions, potassium ions, or both, can also be employed in the compositions. These may facilitate downstream nucleic acid amplification of the sample, for example by PCR, such as qPCR, LAMP, and the like. The amount of magnesium ions, when employed, is typically 1 mM to 15 mM. The amount of potassium ions, when employed, is typically 5 mM to 500 mM, such as 20 mM to 60 mM. Particularly magnesium ion is included as a component of a magnesium salt, such as magnesium sulfate or a hydrate thereof, and more particularly magnesium sulfate heptahydrate.

Optionally, the composition can further include one or more additional components. When employed, these are most commonly one or more of an indicator dye, a preservative, an enhancer of a LAMP reaction, an enhancer of a qPCR reaction, or a fluorosurfactant.

When an indicator dye is used, it can be any dye suitable for the intended use, such as suitable for the detection of a microorganism or microorganisms of interest. Many indicator dyes are known in the art, and in principle any of them can be used. One particularly common dye is cresol red. Indicator dyes are not always required; some detection systems do not rely on indicator dyes, and in some cases desired dyes may be added during a downstream processing step.

A variety of preservatives that are suitable for use with biological systems are known, and when one is employed it can be selected by the person of skill in the art depending on the desired end use. One particularly useful preservative is methylisothiazolinone.

Enhancers for facilitating a LAMP or qPCR reaction are also known in the art, and can be selected depending on the desired end use, such as the type of nucleic acid amplification to be employed. Examples include sulfates such as magnesium sulfate and ammonium sulfate, or hydrates thereof, or potassium chloride.

### Kits

The compositions as described above may be prepared in advance for an end-user, or a kit can be provided to an end user who can then prepare the composition from the components of the kit and, optionally, water that is provided by the user. Thus, a kit can include a plurality of zirconium oxide particles, which can be any of the aforementioned particles as described with reference to the composition. The particles of zirconium oxide can be provided as a solid to be dispersed in water, such as deionized water, or it can be provided as a dispersion in water.

A kit can further comprise an organic, iron-chelating reagent having a first affinity constant greater than or equal to 10^{4.2} with respect to ferric iron and a second affinity constant less than 10^{3.8} with respect to magnesium, wherein the first affinity constant and the second affinity constant are determined in 20° C. deionoized water at pH 8.45. Any of the organic, iron-chelating reagents as descried above with reference to the composition can be used. EGTA is most common. The organic, iron-chelating reagent to be dispersed in water, such as deionized water, or it can be provided in water.

The kit can further comprise a buffer. The buffer most commonly has a buffering region at 20° C. that extends from a pH of 7.7 or lower to a pH of 8.2 or higher. Thus, the buffer can typically provide a buffering capacity at in the pH range of the composition, which range is discussed in detail above. The buffer, as is discussed in detail above, is typically zwitterionic and most particularly bicine. The buffer can be provided as a solid to be reconstituted in water, such as deionized water, or it can be dissolved in water.

Similarly, the kit can provide a nanoparticle dispersion stabilizer, at least one of an indicator dye, a preservative, an enhancer of a LAMP reaction, an enhancer of a qPCR reaction, or a fluorosurfactant, and/or a ferric salt. Any of these can be dispersed in water or they can be provided as solids to be later dispersed in water.

Most commonly, at least one of the plurality of zirconium oxide particles, the non-ionic surfactant, and the organic, iron-chelating reagent are disposed in an aqueous liquid that has a pH (when measured at 20° C.) greater than 7.7 and less than 8.45. In particular cases, the pH (when measured at 20° C.) can be greater than 7.7, greater than 7.8, greater than 7.9, or greater than 8.0. In particular, the pH (when measured at 20° C.) can be less than 8.45, less than 8.4, less than 8.3, or less than 8.2. Most commonly, and most particularly, the pH is 7.8-8.3. Any remaining components are most commonly provided as solids to be added to the aqueous liquid later.

### Methods of Use

The compositions and kits as disclosed herein are most commonly used in methods of amplifying nucleic acids. The kits can be used by first combining the components of the kits with each other and/or water, particularly deionized water or reverse-osmosis purified water, to form compositions as disclosed herein. The compositions can then be used according to the general methods known in the art, such as those described in U.S. Pat. No. 10,619,189.

Briefly, a composition as described herein can be contacted with a sample comprising or suspected to comprise a microorganism or a virus to form a mixture. The sample can optionally have been incubated before this contacting step, especially if doing so is necessary to increase the number of microorganisms or viruses. The microorganism or virus can then be lysed to form a lysed mixture. The lysed mixture can then be subjected to a nucleic acid amplification process to amplify one or more nucleic acids that were present in the microorganism or virus.

The lysis step is typically thermal lysis, which is most often accomplished by heating the mixture to 80-115° C. for 5-30 minutes.

The nucleic acid amplification method can be any known method, but is most commonly PCR, such as qPCR, or LAMP. qPCR amplification is known and has been described, for example, in the article “Real-time PCR in the microbiology laboratory” by Mackay, I. in European Society of Clinical Microbiology and Infectious Diseases 2004(190). LAMP amplification is described, for example, in U.S. Pat. No. 9,090,168. The results of amplification, such as LAMP amplification can be detected by known methods, such as those described in the paper “Novel bioluminescent quantitative detection of nucleic acid amplification in real-time” by Gandelman, O. et al PLoS One, November 30;5(11).

### EXAMPLES

The zirconium oxide nanoparticle dispersion (5 weight % in water, ≤100 nm mean particle size (BET), part no. 643122) was obtained from the Sigma Aldrich Company, St. Louis, MO.

Citric acid (part no. C1909), polyvinylpyrrolidone (part no. P5288), TRITON X-100 surfactant (part no. T8787), bicine (part no. B8660), potassium acetate (P1190), potassium hydroxide (part no. 60370), EGTA (part no. 03777), magnesium heptahydrate (part no. 63138), and PROCLIN 950 (part no. 46878-U) were all obtained from the Sigma Aldrich Company.

The pH of compositions was measured at 20° C. using an ACCUMET AE150 benchtop pH meter with an ACCUMET gel-filled polymer body pH/ATC double-junction combination electrode (mercury free) (obtained from Thermo Fisher Scientific, Waltham, MA). Measurements were conducted within 24 hours of the sample preparation.

### Example 1

A suspension composition was prepared by adding each component listed in Table 1 to deionized water in the order specified and mixing. The composition had a pH of 8.1.

TABLE 1

| Order of | | | |
|---|---|---|---|
| Addition | | | |
| to Water | Component | Concentration | |
| 1 | Citric acid | 65.7 | mg/L |
| 2 | TRITON X-100 | 10 | g/L |
| 3 | Polyvinylpyrrolidone (PVP) | 0.43 | g/L |
| | (MW 360,000) | | |
| 4 | Zirconium oxide dispersion | 100 | microliters/L |
| | (5 wt. %) | | |
| 5 | Bicine | 9.82 | g/L (60 mM) |
| 6 | Potassium acetate | 5.92 | g/L |
| 7 | Potassium hydroxide | 1.71 | g/L |
| 8 | EGTA | 2.28 | g/L |
| 9 | Magnesium sulfate heptahydrate | 345 | mg/L |
| 12 | PROCLIN 950 (9.5% solution of | 0.526 | mL/L |
| | 2-methyl-4-isothiazolin-3-one) | | |

### Comparative Example A

The composition was prepared as described in Example 1 with the exception that the citric acid, zirconium oxide dispersion, EGTA, magnesium sulfate heptahydrate components were not included in the composition. The composition had a pH of 8.3.

### Example 2

The composition was prepared by adding each component to deionized water in the order specified in Table 2. The composition had a pH of 8.3. Compared to the Composition of Example 1 (Table 1), this composition (Table 2) had greater concentrations of bicine and potassium hydride, and a lower concentration of potassium acetate to provide increased buffering capacity.

TABLE 2

| Order of | | | |
|---|---|---|---|
| Addition | | | |
| to Water | Component | Concentration | |
| 1 | Citric acid | 65.7 | mg/L |
| 2 | TRITON X-100 | 10 | g/L |
| 3 | Polyvinylpyrrolidone (PVP) | 0.43 | g/L |
| | (MW 360,000) | | |
| 4 | Zirconium oxide dispersion | 100 | microliters/L |
| | (5 wt. %) | | |
| 5 | Bicine | 20.4 | g/L (125 mM) |
| 6 | Potassium acetate | 2.95 | g/L |
| 7 | Potassium hydroxide | 3.37 | g/L |
| 8 | EGTA | 2.28 | g/L |
| 9 | Magnesium sulfate heptahydrate | 345 | mg/L |
| 12 | PROCLIN 950 (9.5% solution of | 0.526 | mL/L |
| | 2-methyl-4-isothiazolin-3-one) | | |

### Comparative Example B

The composition was prepared as described in Example 2 with the exception that the citric acid, zirconium oxide dispersion, EGTA, magnesium sulfate heptahydrate components were not included in the composition. The composition had a pH of 8.3.

### Comparative Example C

A composition was prepared as described in Example 2 of U.S. Pat. No. 10,619,189. The composition had a pH of 8.7.

### Example 3. Loop-Mediated Isothermal Amplification (LAMP)—Bioluminescence Detection Assay using Compositions of Examples 1-2 and Comparative Examples A-C

Raw, ground chicken (32 g) and buffered peptone water enrichment media (BPW-ISO, 162 mL prewarmed to 41.5° C., obtained from the 3M Company, St. Paul, MN) were combined in a Nasco WHIRL-PAK Homogenizer Filter Bag (part no. 01318, obtained from Thermo Fisher

Scientific) and mixed at 230 rpm (revolutions per minute) for 2 minutes. The sample was incubated at 41.5° C. for 6 hours. Aliquots (580 microliters) selected from Compositions of Examples 1-2 and Comparative Examples A-C were individually added to separate 1.1 mL AXYGEN mini-tubes (part no. MTS-11-12-C-R, obtained from Corning Inc., Corning, NY) (i.e., a single composition added per tube). Next, 20 microliters of enriched sample from the homogenizer bag was added to each tube.

For nucleic amplification and bioluminescence detection, each tube was heated in a 100° C. heat block for 15 minutes, cooled to about 40° C., and then a 20 microliter aliquot of the mixture was added to a reaction tube containing a generic matrix control pellet (part no. MDMC96NA, obtained from the 3M Company). Three reaction tubes (n=3) were prepared for each composition.

After dissolving the pellet, each reaction tube was analyzed using a 3M Molecular Detection Instrument (part no. MDS 100; obtained from the 3M Company) with recording of the bioluminescence signal according to the manufacturer's instructions. The maximum bioluminescence signal (in relative light units (RLU)) and the reaction time at which the maximum bioluminescence signal occurred were recorded. The results are reported in Tables 3-5 as the average of 3 trials.

TABLE 3

| | Time to the Maximum | Maximum |
|---|---|---|
| | Bioluminescence Signal | Bioluminescence Signal |
| Composition | (minutes) | (RLU) |
| Example 1 | 14.9 | 192,053 |
| Comparative | 21.3 | 204,383 |
| Example A | | |

TABLE 4

| | Time to the Maximum | Maximum |
|---|---|---|
| | Bioluminescence Signal | Bioluminescence Signal |
| Composition | (minutes) | (RLU) |
| Example 2 | 13.5 | 256,767 |
| Comparative | 21.4 | 196,953 |
| Example B | | |

TABLE 5

| | Time to the Maximum | Maximum |
|---|---|---|
| | Bioluminescence Signal | Bioluminescence Signal |
| Composition | (minutes) | (RLU) |
| Comparative | 15.4 | 160,841 |
| Example C | | |

### Example 4. LAMP—Bioluminescence Detection Assay using Compositions with Different pH Values

The pH of the composition prepared according to the procedure of Example 1 (Table 1) was adjusted with varying amounts of glacial acetic acid to provide five separate compositions having a pH of either 7.4, 7.6, 7.8, 8.0, or 8.2. The reaction time at which the maximum bioluminescence signal occurred was determined according to the procedure of Example 3. The results are reported in Table 6 as the average of 3 trials.

TABLE 6

| | Time to the Maximum |
|---|---|
| pH of | Bioluminescence Signal |
| Composition | (minutes) |
| 8.2 | 13.8 |
| 8.0 | 14.8 |
| 7.8 | 14.8 |
| 7.6 | 23.8 |
| 7.4 | 33.3 |

1. An aqueous composition comprising:
zirconium oxide particles;
a surfactant at a concentration greater than or equal to 0.005% (mass/volume);
an organic, iron-chelating reagent having a first affinity constant greater than or equal to 10^{4.2} with respect to ferric iron and a second affinity constant less than 10^{3.8} with respect to magnesium, wherein the first affinity constant and the second affinity constant are determined in 20° C. deionized water at pH 8.45; and
a buffer, the buffer optionally having a concentration of 40 mM or greater, further optionally having a concentration of 40 mM to about 200 mM, and still further optionally having a concentration of 40 mM to 150 mM;
wherein the composition has a pH greater than 7.7 and less than 8.45, optionally 7.8-8.3 when measured at 20° C.

2. The aqueous buffer composition of claim 1, wherein the buffer comprises at least one zwitterionic compound.

3. The aqueous buffer composition of claim 2, wherein the at least one zwitterionic compound comprises bicine.

4. The aqueous composition of claim 1, further comprising citric acid or a salt thereof.

5. The aqueous composition of claim 1, further comprising a magnesium salt, optionally wherein the magnesium salt is magnesium sulfate or a hydrate thereof, and further optionally magnesium sulfate heptahydrate.

6. The aqueous composition of claim 1, wherein the organic, iron-chelating reagent comprises ethylene glycol-bis(2-aminoethylether)-N,N,N′,N′-tetracetic acid, N,N′,N′,N′-tetrakis(2-pyridinylmethyl)ethan-1,2-diamine, 1,2-bis(O-aminophenoxy)ethane-N,N,N,N′-tetracetic acid, N-(2-hydroetoxyethyl) ethylenediamine-N,N′,N′-triacetic acid, a salt of any of the foregoing, or a hydrate of any of the foregoing.

7. The aqueous composition of any of claim 1, further comprising ferric ion.

8. The aqueous composition of claim 1, wherein the zirconium oxide particles have a mean particle size of no more than 500 nm, optionally no more than 250 nm, optionally no more than, in each case as measured by photon correlation spectroscopy as described herein.

9. The aqueous composition of claim 1, further comprising at least one of an indicator dye, a preservative, an enhancer of a LAMP reaction, an enhancer of a qPCR reaction, or a fluorosurfactant.

10. A method of amplifying nucleic acids comprising,
a) contacting a composition with a sample comprising a microorganism or a virus to form a mixture;
b) lysing the microorganism or the virus in the mixture to form a lysed mixture; and
c) subjecting at least a portion of the lysed mixture to a nucleic acid amplification process.

11. The method of claim 10, further comprising a step of incubating the composition comprising a microorganism or a virus in a growth medium prior to step a).

12. The method of claim 10, wherein the nucleic acid amplification process comprises a polymerase chain reaction (PCR) or a loop-mediated isothermal amplification (LAMP).

13. The method of claim 12, wherein the PCR is qPCR.

14. The method of claim 10, wherein the lysing step comprises thermal lysis, the thermal lysis optionally comprising heating the mixture to 80° C.-115° C. for 5-30 minutes.

15. A kit, comprising
a plurality of zirconium oxide particles;
a non-ionic surfactant;
an organic, iron-chelating reagent having a first affinity constant greater than or equal to 10^{4.2} with respect to ferric iron and a second affinity constant less than 10^{3.8} with respect to magnesium, wherein the first affinity constant and the second affinity constant are determined in 20° C. deionized water at pH 8.45;
a buffer, the buffer having a buffering region at 20° C. that extends from a pH of 7.8 or lower to a pH of 8.2 or higher.

16. The kit of claim 15, further comprising a nanoparticle dispersion stabilizer.

17. The kit of claim 15, wherein the non-ionic surfactant has a hydrophilic-lipophilic balance of about 11 to about 16.

18. The kit of claim 15, wherein at least one of the plurality of zirconium oxide particles, the non-ionic surfactant, and the organic, iron-chelating reagent are disposed in an aqueous liquid that has a pH greater than 7.7 and less than 8.45, optionally 7.8-8.3 when measured at 20° C.

19. The kit of claim 15, further comprising an indicator dye, a preservative, an enhancer of a LAMP reaction, an enhancer of a qPCR reaction, or a fluorosurfactant, or any combination thereof.

20. The kit of claim 15, wherein the kit further comprises a ferric salt.
